SEQUENCE LISTING (10 sequences)

SEQ ID NO: 1 - protein, 120 aa, Artificial Sequence
  Synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVS

SEQ ID NO: 2 - protein, 109 aa, Artificial Sequence
  Synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRT

SEQ ID NO: 3 - protein, 10 aa, Artificial Sequence
  Synthetic construct
GFSLIGYDLN

SEQ ID NO: 4 - protein, 16 aa, Artificial Sequence
  Synthetic construct
IIWGDGTTDYNSAVKS

SEQ ID NO: 5 - protein, 13 aa, Artificial Sequence
  Synthetic construct
GGYWYATSYYFDY

SEQ ID NO: 6 - protein, 11 aa, Artificial Sequence
  Synthetic construct
RASQSISNNLN

SEQ ID NO: 7 - protein, 7 aa, Artificial Sequence
  Synthetic construct
YTSRFHS

SEQ ID NO: 8 - protein, 9 aa, Artificial Sequence
  Synthetic construct
QQEHTLPYT

SEQ ID NO: 9 - protein, 447 aa, Artificial Sequence
  Synthetic construct
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, Artificial Sequence
  Synthetic construct
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0258664 A1
(43) Pub. Date: Aug. 17, 2023

(54) ENDOTOXIN DETECTION

(71) Applicants: Pfizer Inc., New York, NY (US); Eli Lilly and Company, Indianapolis, IN (US)

(72) Inventors: Matthew Christopher Bedwell, Indianapolis, IN (US); Ned Michael Mozier, Chesterfield, MO (US); Tammy Thurman, Chesterfield, MO (US); Robert Erwin Warburton, JR., Indianapolis, IN (US)

(73) Assignees: Pfizer Inc., New York, NY (US); Eli Lilly and Company, Indianapolis, IN (US)

(21) Appl. No.: 17/787,015

(22) PCT Filed: Dec. 18, 2020
(86) PCT No.: PCT/IB2020/062212

§ 371 Date: Feb. 6, 2023

Related U.S. Application Data
(60) Provisional application No. 63/117,201, filed on Nov. 23, 2020.
(60) Provisional application No. 62/951,496, filed on Dec. 20, 2019.

(51) Int. Cl. G01N 33/92; G01N 33/563; G01N 33/569

(52) U.S. Cl. CPC G01N 33/92; G01N 33/563; G01N 33/56911; G01N 2400/50

(57) ABSTRACT

Provided herein are methods and compositions for overcoming Low Endotoxin Recovery (LER) and unmasking endotoxins. The compositions and methods provided herein may be used to prepare samples such as drug products for endotoxin testing.

### RELATED APPLICATIONS

The present invention is a national stage filing under 35 U.S.C. § 371 of international PCT application PCT/IB2020/062212, filed Dec. 18, 2020, which claims priority under 35 U.S.C. § 119(e) to U.S. provisional patent application, U.S. Ser. No. 62/951,496, filed Dec. 20, 2019, and U.S. provisional patent application, U.S. Ser. No. 63/117,201, filed Nov. 23, 2020 the entire contents of each of which are incorporated herein by reference.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Jun. 17, 2022, is named P113970007US00-SEQ-ROS and is 9,667 bytes in size.

### FIELD

The present invention relates to methods and compositions for improving the detection of endotoxin in samples of interest. In particular, provided herein are methods and compositions for unmasking endotoxins. Methods and compositions provided herein may be used, for example, to improve the detection of endotoxin in pharmaceutical formulations.

### BACKGROUND

Endotoxins are the major component of the cell wall of gram-negative bacteria. Endotoxin is also known as lipopolysaccharide (LPS), and is composed of lipid A, the core polysaccharide, and the O-antigen polysaccharide. Introduction of endotoxin into humans or other subjects can trigger an inflammatory response. The inflammatory response can be severe, and result in shock or even death of the subject. Because of the risks associated with accidental administration of endotoxin to a subject, compositions such as pharmaceutical formulations must be tested for endotoxin contamination before being released for administration to subjects.

Various methods are known in the art for detecting endotoxin, such as the rabbit pyrogen test (RPT) and the Limulus Amebocyte Lysate (LAL) assay. The LAL assay is generally preferred over the RPT assay due to greater specificity and sensitivity. The LAL assay involves an enzyme cascade that is triggered in response to endotoxin; the first enzyme in this cascade is Factor C enzyme, which binds to endotoxin/LPS. Different versions of the LAL assay exist, including chromogenic, turbidimetric, and gel clot LAL assays. Historically, LAL assays used blood cells (amebocytes) from the blood of horseshoe crabs. Alternatives to LAL assays are now available which use recombinant proteins instead of horseshoe crab blood cells (e.g. PyroGene™ Recombinant Factor C Assay, Lonza).

More recently, a phenomenon now commonly referred to as “Low Endotoxin Recovery” (LER) was identified. Specifically, it was observed that in some circumstances, endotoxin spiked into a neat (nearly undiluted) samples could not be adequately recovered when tested for endotoxin. Thus, LER refers to a situation where a sample contains spiked endotoxin, but the endotoxin cannot be efficiently detected in the sample. In an LER situation, endotoxin in a sample may be described as being “masked”. Endotoxin is “masked” when one or more macromolecules (e.g. proteins) or small molecules (e.g. metal ions or detergents) associate with endotoxin in such a way to prevent Factor C or other component of an LAL assay from binding to the endotoxin, and thus masking/shielding endotoxin from detection in the endotoxin assay.

While various factors that may lead to LER/endotoxin masking have been identified, the phenomenon is still poorly understood and unpredictable. In addition, while some methods for overcoming LER and “unmasking” endotoxin have been developed, some samples of interest exhibit LER that cannot be overcome by known methods for addressing LER.

Accordingly, there is a need for new and improved methods for addressing LER and unmasking endotoxins.

### SUMMARY

Provided herein are methods and compositions for unmasking endotoxin and overcoming situations of Low Endotoxin Recovery (LER) related to endotoxin detection assays.

In some embodiments, provided herein is a biphasic mixture comprising a first liquid phase and a second liquid phase, wherein: a) the first liquid phase is a solution comprising a molecule of interest and masked endotoxin; and b) the second liquid phase is liquid phase 1-dodecanol; wherein the second liquid phase floats on the first liquid phase. Optionally, the biphasic mixture does not contain bovine serum albumin (BSA).

In some embodiments, provided herein is a biphasic mixture comprising a first liquid phase and a second liquid phase, wherein: a) the first liquid phase is a solution comprising a molecule of interest and masked endotoxin; and b) the second liquid phase is liquid phase 1-dodecanol. Optionally, the biphasic mixture does not contain bovine serum albumin (BSA).

In some embodiments, provided herein is a biphasic mixture comprising a liquid phase and a solid phase, wherein: a) the liquid phase is a solution comprising a molecule of interest and masked endotoxin; and b) the solid phase is solid 1-dodecanol; and wherein the solid phase floats on the liquid phase. Optionally, the biphasic mixture does not contain bovine serum albumin (BSA).

In some embodiments, provided herein is a biphasic mixture comprising a liquid phase and a solid phase, wherein: a) the liquid phase is a solution comprising a molecule of interest and masked endotoxin; and b) the solid phase is solid 1-dodecanol. Optionally, the biphasic mixture does not contain bovine serum albumin (BSA).

In some embodiments, provided herein is a method of unmasking endotoxin in a solution comprising a molecule of interest and masked endotoxin, the method comprising: a) adding liquid phase 1-dodecanol to a solution containing the molecule of interest and the masked endotoxin; and b) cooling the liquid phase 1-dodecanol and solution containing the molecule of interest to a temperature below 24° C., such that the 1-dodecanol solidifies and there is a remaining liquid portion and the solidified 1-dodecanol. Optionally, the remaining liquid portion is “Liquid A”, and the method further comprises adding a solution comprising Ca²⁺ or Mg²⁺ ions to Liquid A, to generate a mixture (“Liquid B”) comprising Ca²⁺ or Mg²⁺ ions and Liquid A. Optionally, the method further comprises incubating Liquid B. Optionally, the method further comprises, after incubating Liquid B, vortexing Liquid B. Optionally, the method further comprises diluting a portion of Liquid B into a buffer comprising MgSO₄ to yield a sample ready for endotoxin testing, wherein the previously masked endotoxin is unmasked in the sample ready for endotoxin testing.

In some embodiments, provided herein is a method of unmasking endotoxin in a solution comprising a molecule of interest and masked endotoxin, the method comprising: a) adding liquid phase 1-dodecanol to a solution containing the molecule of interest and the masked endotoxin; b) cooling the liquid phase 1-dodecanol and solution containing the molecule of interest to a temperature below 24° C., such that the 1-dodecanol solidifies and there is a remaining liquid portion (“Liquid A”) and the solid 1-dodecanol; c) adding a solution comprising Ca²⁺ or Mg²⁺ ions to Liquid A, to generate a mixture (“Liquid B”) comprising Ca²⁺ or Mg²⁺ ions and Liquid A; d) incubating Liquid B; e) vortexing Liquid B; and f) diluting a portion of Liquid B into a buffer comprising MgSO₄ to yield a sample ready for endotoxin testing; wherein the previously masked endotoxin is unmasked in the sample of step f).

In some embodiments, provided herein is a method of unmasking endotoxin in a solution comprising a molecule of interest and masked endotoxin, the method comprising: a) adding liquid phase 1-dodecanol to a solution containing the molecule of interest and the masked endotoxin; and b) cooling the liquid phase 1-dodecanol and solution containing the molecule of interest to a temperature below 24° C., such that the 1-dodecanol solidifies and there is a remaining liquid portion and the solidified 1-dodecanol. Optionally, the remaining liquid portion is “Liquid A”, and the method further comprises adding a dispersant solution to Liquid A, to generate a mixture (“Liquid B”) comprising dispersant and Liquid A. Optionally, the method further comprises incubating Liquid B. Optionally, the method further comprises, after incubating Liquid B, vortexing Liquid B. Optionally, the method further comprises diluting a portion of Liquid B into a buffer comprising Ca²⁺ or Mg²⁺ ions to yield a sample ready for endotoxin testing, wherein the previously masked endotoxin is unmasked in the sample ready for endotoxin testing.

In some embodiments, provided herein is a method of unmasking endotoxin in a solution comprising a molecule of interest and masked endotoxin, the method comprising: a) adding liquid phase 1-dodecanol to a solution containing the molecule of interest and the masked endotoxin; b) cooling the liquid phase 1-dodecanol and solution containing the molecule of interest to a temperature below 24° C., such that the 1-dodecanol solidifies and there is a remaining liquid portion (“Liquid A”) and the solid 1-dodecanol; c) adding a dispersant solution to Liquid A, to generate a mixture (“Liquid B”) comprising dispersant and Liquid A; d) incubating Liquid B; e) vortexing Liquid B; and f) diluting a portion of Liquid B into a buffer comprising Ca²⁺ or Mg²⁺ ions to yield a sample ready for endotoxin testing; wherein the previously masked endotoxin is unmasked in the sample of step f).

In some embodiments, provided herein is a method of unmasking endotoxin in a solution comprising a molecule of interest and masked endotoxin, the method comprising: a) adding liquid phase 1-dodecanol to a solution containing the molecule of interest and the masked endotoxin; b) cooling the liquid phase 1-dodecanol and solution containing the molecule of interest to a temperature below 24° C., such that the 1-dodecanol solidifies and there is a remaining liquid portion (“Liquid A”) and the solid 1-dodecanol; and c) diluting a portion of Liquid A into a buffer comprising Ca²⁺ or Mg²⁺ ions to yield a sample ready for endotoxin testing; wherein the previously masked endotoxin is unmasked in the sample of step c.

In some embodiments, provided herein is a method of unmasking endotoxin in a solution comprising a molecule of interest and masked endotoxin, the method comprising: a) adding liquid phase 1-dodecanol to a solution containing the molecule of interest and the masked endotoxin; b) cooling the liquid phase 1-dodecanol and solution containing the molecule of interest to a temperature below 24° C., such that the 1-dodecanol solidifies and there is a remaining liquid portion (“Liquid A”) and the solid 1-dodecanol; c) adding a dispersant solution to Liquid A, to generate a mixture (“Liquid B”) comprising dispersant and Liquid A; and d) diluting a portion of Liquid B into a buffer comprising Ca²⁺ or Mg²⁺ ions to yield a sample ready for endotoxin testing; wherein the previously masked endotoxin is unmasked in the sample of step d.

In some embodiments of a biphasic mixture or method provided herein, the molecule of interest is a protein. Optionally, the protein is an antibody. Optionally, the antibody is tanezumab.

In some embodiments of a biphasic mixture or method provided herein, the solution comprising a molecule of interest is a tanezumab drug product formulation, such as described in WO2010/032220, herein incorporated by reference for all purposes. Optionally, the formulation is a liquid formulation and comprises tanezumab antibody at a concentration of about 2.5 mg/ml, 5 mg/ml, 10 mg/ml or 20 mg/ml; and optionally, a histidine buffer. Optionally, the formulation further comprises a surfactant which may be polysorbate 20. In some embodiments, the formulation further comprises trehalose dehydrate or sucrose. In some embodiments, the formulation further comprises a chelating agent, which may be EDTA; in some embodiments disodium EDTA. In some embodiments, the formulation is of pH 6.0±0.3.

In some embodiments of a biphasic mixture or method provided herein, the solution comprising a molecule of interest (e.g. tanezumab drug product formulation) is an aqueous solution.

In some embodiments of a biphasic mixture or method provided herein, the solution comprising a molecule of interest is a tanezumab drug product formulation, wherein the formulation comprises about 2.5 mg/ml, 5 mg/ml, 10 mg/ml or 20 mg/ml tanezumab; about 10 mM histidine buffer; about 84 mg/ml trehalose dehydrate; about 0.1 mg/ml Polysorbate 20; about 0.05 mg/ml disodium EDTA; wherein the formulation is of a pH 6.0±0.3.

In some embodiments of a biphasic mixture or method provided herein comprising tanezumab the solution comprises 2.5 mg/ml, 5 mg/ml, 10 mg/ml, or 20 mg/ml tanezumab. Optionally, the formulation has a total volume of about 1 ml. Optionally, the formulation is contained in a glass or plastic vial or syringe. Optionally, the formulation is contained in a pre-filled glass or plastic vial or syringe.

In some embodiments of a biphasic mixture or method provided herein comprising 1-dodecanol, the 1-dodecanol is ≥98% or ≥99% pure 1-dodecanol.

In some embodiments of a biphasic mixture or method provided herein the ratio of microliters of 1-dodecanol to microliters of solution comprising the molecule of interest and masked endotoxin is between a) 0.2 microliters 1-dodecanol per 10 microliters solution and b) 2 microliters 1-dodecanol per 10 microliters solution. Optionally, the ratio of microliters of 1-dodecanol to microliters of solution comprising the molecule of interest and masked endotoxin is between a) 0.5 microliters 1-dodecanol per 10 microliters solution and b) 1.5 microliters 1-dodecanol per 10 microliters solution. Optionally, the ratio of microliters of 1-dodecanol to microliters of solution comprising the molecule of interest and masked endotoxin is 1 microliter 1-dodecanol per 9 microliters solution (e.g. 1 microliter 1-dodecanol+9 microliters solution).

In some embodiments of a biphasic mixture or method provided herein comprising 1-dodecanol and a solution comprising the molecule of interest, the mixture contains between about 5%-11% (v/v) 1-dodecanol/solution comprising the molecule of interest. Optionally, the mixture contains about 10% (v/v) 1-dodecanol/solution comprising the molecule of interest.

In some embodiments of a method provided herein involving cooling the liquid phase 1-dodecanol and solution containing the molecule of interest to a temperature below 24° C., such that the 1-dodecanol solidifies and there is a remaining liquid portion (“Liquid A”) and the solid 1-dodecanol, the liquid phase 1-dodecanol and solution containing the molecule of interest are cooled in an ice water bath or cooling block. Optionally, the liquid phase 1-dodecanol and solution containing the molecule of interest are cooled for a period of time sufficient for the liquid phase 1-dodecanal to solidify. Optionally, the liquid phase 1-dodecanol and solution containing the molecule of interest are cooled for about 0.5-20 minutes. Optionally, the liquid phase 1-dodecanol and solution containing the molecule of interest are cooled for about 1, 2, or 3 minutes.

In some embodiments of a method provided herein involving a dispersant solution, the dispersant solution is or comprises PYROSPERSE™.

In some embodiments of a method provided herein involving a dispersant solution, the dispersant solution is a solution comprising cations. In some embodiments, a dispersant solution comprising cations comprises divalent cations. In some embodiments, a dispersant solution comprising divalent cations comprises Ca²⁺ or Mg²⁺ ions. In some embodiments, a dispersant solution comprising Ca²⁺ or Mg²⁺ ions comprises between 1M-2M Ca²⁺ or Mg²⁺ ions. In some embodiments, a dispersant solution comprising Ca²⁺ or Mg²⁺ ions comprises about 1.5M Ca²⁺ or Mg²⁺ ions. In some embodiments, a dispersant solution comprising Ca²⁺ or Mg²⁺ ions comprises CaCl₂. In some embodiments, a dispersant solution comprising Ca²⁺ or Mg²⁺ ions comprises 1.5 CaCl₂.

In some embodiments of a method provided herein involving a dispersant solution, the dispersant solution comprises a chaotropic agent. Chaotropic agents are molecules that can disrupt non-covalent forces (e.g. hydrogen bonds, hydrophobic effects) between and within molecules, and may solubilize and/or denature macromolecules such as proteins. Exemplary chaotropic agents include guanidinium chloride, urea, thiourea, sodium dodecyl sulfate, lithium perchlorate, lithium acetate, magnesium chloride, phenol, 2-propanol, n-butanol, and ethanol.

In some embodiments, a dispersant solution comprises guanidinium chloride (GuHCl), also known as guanidine hydrochloride. Optionally, a dispersant solution comprising guanidine hydrochloride comprises between 0.5-8 M, 1-8 M, 2-8 M, 4-8 M, or 6-8 M guanidine hydrochloride. Optionally, a dispersant solution comprising guanidine hydrochloride comprises about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, or 8 M guanidine hydrochloride. Optionally, a dispersant solution comprising guanidine hydrochloride comprises between about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, or 7.5 M guanidine hydrochloride and 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, or 8 M guanidine hydrochloride, wherein the first value is smaller than the second value.

In some embodiments, a dispersant solution comprises urea. Optionally, a dispersant solution comprising urea comprises between 0.5-8 M, 1-8 M, 2-8 M, 4-8 M, or 6-8 M urea. Optionally, a dispersant solution comprising urea comprises about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M urea. Optionally, a dispersant solution comprising urea comprises between about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, or 7.5 M urea and 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M urea, wherein the first value is smaller than the second value.

In some embodiments, a dispersant solution comprises thiourea. Optionally, a dispersant solution comprising thiourea comprises between 0.5-4 M, 1-4 M, 2-4 M, or 3-4 M thiourea. Optionally, a dispersant solution comprising thiourea comprises about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, or 4 M thiourea. Optionally, a dispersant solution comprising thiourea comprises between about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, or 3.5 M, thiourea and 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M thiourea, wherein the first value is smaller than the second value.

In some embodiments, a dispersant solution comprises lithium perchlorate. Optionally, a dispersant solution comprising lithium perchlorate comprises between 0.5-8 M, 1-8 M, 2-8 M, 4-8 M, or 6-8 M lithium perchlorate. Optionally, a dispersant solution comprising lithium perchlorate comprises about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M lithium perchlorate. Optionally, a dispersant solution comprising lithium perchlorate comprises between about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, or 7.5 M lithium perchlorate and 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M lithium perchlorate, wherein the first value is smaller than the second value.

In some embodiments, a dispersant solution comprises lithium acetate. Optionally, a dispersant solution comprising lithium acetate comprises between 0.5-8 M, 1-8 M, 2-8 M, 4-8 M, or 6-8 M lithium acetate. Optionally, a dispersant solution comprising lithium acetate comprises about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M lithium acetate. Optionally, a dispersant solution comprising lithium acetate comprises between about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, or 7.5 M lithium acetate and 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M lithium acetate, wherein the first value is smaller than the second value.

In some embodiments, a dispersant solution comprises magnesium chloride. Optionally, a dispersant solution comprising magnesium chloride comprises between 0.5-8 M, 1-8 M, 2-8 M, 4-8 M, or 6-8 M magnesium chloride. Optionally, a dispersant solution comprising magnesium chloride comprises about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M magnesium chloride. Optionally, a dispersant solution comprising magnesium chloride comprises between about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, or 7.5 M magnesium chloride and 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M magnesium chloride, wherein the first value is smaller than the second value.

In some embodiments, a dispersant solution comprises sodium dodecyl sulfate (SDS). Optionally, a dispersant solution comprising sodium dodecyl sulfate comprises between 0.5-8 M, 1-8 M, 2-8 M, 4-8 M, or 6-8 M sodium dodecyl sulfate. Optionally, a dispersant solution comprising sodium dodecyl sulfate comprises about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M sodium dodecyl sulfate. Optionally, a dispersant solution comprising sodium dodecyl sulfate comprises between about 0.1 M, 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, or 7.5 M sodium dodecyl sulfate and 0.2 M, 0.3 M, 0.4 M, 0.5 M, 0.6 M, 0.7 M, 0.8 M, 0.9 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M sodium dodecyl sulfate, wherein the first value is smaller than the second value.

In some embodiments, more than one type of chaotropic agent may be present in a dispersant solution

In some embodiments of a method provided herein involving adding a dispersant solution to Liquid A, to generate a mixture (“Liquid B”), the ratio of microliters of dispersant solution to Liquid A is between a) 0.5 microliters dispersant solution per 100 microliters Liquid A and b) 5 microliters dispersant solution per 100 microliters Liquid A. Optionally, the ratio of microliters dispersant solution to Liquid A is 3 microliters dispersant solution per 100 microliters Liquid A.

In some embodiments of a method provided herein involving adding a dispersant solution to Liquid A, to generate a mixture (“Liquid B”), Liquid B contains between about 0.5%-4% (v/v) dispersant/Liquid A. Optionally, Liquid B contains about 3% (v/v) dispersant/Liquid A.

In some embodiments of a method provided herein, no dispersant solution (0 microliters) is added to Liquid A.

In some embodiments of a method provided herein involving adding a solution comprising Ca²⁺ or Mg²⁺ ions to Liquid A, to generate a mixture (“Liquid B”), the solution comprising Ca²⁺ or Mg²⁺ ions comprises a concentration of Ca²⁺ or Mg²⁺ ions of 1M, 2M, or a value between 1M and 2M. Optionally, the solution comprising Ca²⁺ or Mg²⁺ ions comprises a concentration of Ca²⁺ or Mg²⁺ ions of 1.5 M. Optionally, the ratio of microliters of 1.5M Ca²⁺ or Mg²⁺ ion solution to Liquid A is between a) 0.5 microliters 1.5M Ca²⁺ or Mg²⁺ ion solution per 100 microliters Liquid A and b) 5 microliters 1.5M Ca²⁺ or Mg²⁺ ion solution per 100 microliters Liquid A. Optionally, the ratio of microliters of 1.5M Ca²⁺ or Mg²⁺ ion solution to Liquid A is 3 microliters 1.5M Ca²⁺ or Mg²⁺ ions solution per 100 microliters Liquid A.

In some embodiments of a method provided herein involving incubating Liquid B, Liquid B is incubated at ambient temperature. Optionally, Liquid B is incubated for 0.5-20 minutes. Optionally, Liquid B is incubated for 8-10 minutes. Optionally, Liquid B is incubated for an amount of time ranging from less than 1 minute to greater than 10 minutes.

In some embodiments of a method provided herein involving Liquid B, Liquid B is not incubated. For example, optionally Liquid B is not incubated before a vortexing step or before diluting a portion of Liquid B into a diluent buffer.

In some embodiments of a method provided herein involving vortexing Liquid B, Liquid B is vortexed for 0.1-5 minutes. Optionally, Liquid B is vortexed for 1 minute. Optionally, Liquid B is vortexed for less than 1 minute.

In some embodiments of a method provided herein involving a buffer comprising Ca²⁺ or Mg²⁺ ions, the buffer comprises 1-10, 1-20, 1-50, 1, 5, 10, or 20 mM of Ca²⁺ or Mg²⁺ ions. In some embodiments of a method provided herein involving a buffer comprising Ca²⁺ or Mg²⁺ ions, the buffer comprises Mg²⁺ ions. Optionally, the buffer comprises 1-10 mM Mg²⁺ ions. Optionally, the buffer comprises 1-20 mM Mg²⁺ ions. Optionally, the buffer comprises 1-50 mM Mg²⁺ ions. Optionally, the buffer comprises 1 mM Mg²⁺ ions. Optionally, the buffer comprises 5 mM Mg²⁺ ions. Optionally, the buffer comprises 10 mM Mg²⁺ ions. Optionally, the buffer comprises 20 mM Mg²⁺ ions. In some embodiments of a method provided herein involving a buffer comprising Ca²⁺ or Mg²⁺ ions, the buffer comprises MgSO₄. Optionally, the buffer comprises 1-50 mM MgSO₄. Optionally, the buffer comprises 1-10 mM MgSO₄. Optionally, the buffer comprises 1 mM MgSO₄. Optionally, the buffer comprises 5 mM MgSO₄. Optionally, the buffer comprises 10 mM MgSO₄. Optionally, the buffer comprises 20 mM MgSO₄.

In some embodiments of a method provided herein involving diluting a portion of Liquid B into a buffer comprising Ca²⁺ or Mg²⁺ ions to yield a sample ready for endotoxin testing the buffer comprises 1-10, 1-20, 1-50, 1, 5, 10, or 20 mM of Ca²⁺ or Mg²⁺ ions. Optionally, the buffer comprising Ca²⁺ or Mg²⁺ ions comprises 1-10 mM Mg²⁺ ions. Optionally, the buffer comprises 1-20 mM Mg²⁺ ions. Optionally, the buffer comprises 1-50 mM Mg²⁺ ions. Optionally, the buffer comprises 1 mM Mg²⁺ ions. Optionally, the buffer comprises 5 mM Mg²⁺ ions. Optionally, the buffer comprises 10 mM Mg²⁺ ions. Optionally, the buffer comprises 20 mM Mg²⁺ ions. Optionally, the buffer further comprises 2-100 mM Tris, pH 6.8-7.6. Optionally, the buffer further comprises 2-20 mM Tris, pH 6.8-7.6. Optionally, the buffer further comprises 1-100 mM Tris, pH 6.8-7.6. Optionally, the buffer further comprises 1-20 mM Tris, pH 6.8-7.6. Optionally, the buffer further comprises 2 mM Tris, pH 6.8-7.6. Optionally, the buffer further comprises 5 mM Tris, pH 6.8-7.6. Optionally, the buffer further comprises 10 mM Tris, pH 6.8-7.6. Optionally, the buffer further comprises 20 mM Tris, pH 6.8-7.6. Optionally, the portion of Liquid B is diluted into the buffer comprising Ca²⁺ or Mg²⁺ ions at a ratio between a) 1 microliter Liquid B per 100 microliters Ca²⁺ or Mg²⁺ ions buffer and b) 1 microliter Liquid B per 3000 microliters Ca²⁺ or Mg²⁺ ions buffer. Optionally, the portion of Liquid B is diluted into the buffer comprising Ca²⁺ or Mg²⁺ ions at a ratio between a) 1 microliter Liquid B per 100 microliters Ca²⁺ or Mg²⁺ ions buffer and b) 1 microliter Liquid B per 2500 microliters Ca²⁺ or Mg⁺ ions buffer. Optionally, the portion of Liquid B is diluted into the buffer comprising Ca²⁺ or Mg²⁺ ions at a ratio of 1 microliter Liquid B per about 100, 200, 295, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 microliters Ca²⁺ or Mg²⁺ ions buffer. Optionally, the portion of Liquid B is diluted into the buffer comprising Ca²⁺ or Mg²⁺ ions at a ratio of 1 microliter Liquid B per about 100, 295, 2000, or 2500 microliters Ca²⁺ or Mg²⁺ ions buffer.

In some embodiments of a method provided herein involving diluting a portion of Liquid B into a buffer comprising MgSO₄ to yield a sample ready for endotoxin testing, the buffer comprising MgSO₄ comprises 1-50 mM MgSO₄. Optionally, the buffer comprising MgSO₄ comprises 10 mM MgSO₄. Optionally, the buffer comprising MgSO₄ further comprises 2-100 mM Tris, pH 6.8-7.6. Optionally, the buffer comprising MgSO₄ further comprises 20 mM Tris, pH 6.8-7.6. Optionally, the portion of Liquid B is diluted into the buffer comprising MgSO₄ at a ratio between a) 1 microliter Liquid B per 100 microliters MgSO₄ buffer and b) 1 microliter Liquid B per 3000 microliters MgSO₄ buffer. Optionally, the portion of Liquid B is diluted into the buffer comprising MgSO₄ at a ratio of 1 microliter Liquid B per 2000 microliters MgSO₄ buffer.

In some embodiments, provided herein is a method of unmasking endotoxin in a solution comprising a molecule of interest and masked endotoxin, the method comprising: a) adding 100 microliters liquid phase 1-dodecanol to 900 microliters of a solution containing the molecule of interest and the masked endotoxin; b) cooling the liquid phase 1-dodecanol and solution containing the molecule of interest in an ice water bath or cooling block for 2 minutes to a temperature below 24° C., such that the 1-dodecanol solidifies and there is a remaining liquid portion (“Liquid A”) and the solid 1-dodecanol; c) adding 30 microliters of a solution comprising 1.5 M Ca²⁺ or Mg²⁺ ions to Liquid A, to generate a mixture (“Liquid B”) comprising Ca²⁺ or Mg²⁺ ions and Liquid A; d) incubating Liquid B at ambient temperature for 8-10 minutes; e) after incubating Liquid B, vortexing Liquid B for 1 minute; f) diluting a portion of Liquid B from step e) into a buffer comprising 10 mM MgSO₄ for a dilution of 1:2000, to yield a sample ready for endotoxin testing; wherein the previously masked endotoxin is unmasked in the sample of step f).

In some embodiments, provided herein is a method of unmasking endotoxin in a solution comprising a molecule of interest and masked endotoxin, the method comprising: a) adding 100 microliters liquid phase 1-dodecanol to 900 microliters of a solution containing the molecule of interest and the masked endotoxin; b) cooling the liquid phase 1-dodecanol and solution containing the molecule of interest in an ice water bath or cooling block to a temperature below 24° C., such that the 1-dodecanol solidifies and there is a remaining liquid portion (“Liquid A”) and the solid 1-dodecanol; c) adding 30 microliters of a dispersant solution to Liquid A, to generate a mixture (“Liquid B”) comprising dispersant solution and Liquid A; d) incubating Liquid B at ambient temperature; e) after incubating Liquid B, vortexing Liquid B; f) diluting a portion of Liquid B from step e) into a buffer comprising 10 mM MgSO₄ for a dilution of 1:2000, to yield a sample ready for endotoxin testing; wherein the previously masked endotoxin is unmasked in the sample of step f).

In some embodiments of a method provided herein involving a solution comprising Ca²⁺ or Mg²⁺ ions, the solution comprising Ca²⁺ or Mg²⁺ ions is PYROSPERSE™ solution.

In some embodiments of a method provided herein involving a solution comprising Ca²⁺ or Mg²⁺ ions, the solution comprising Ca²⁺ or Mg²⁺ comprises 1.5 M CaCl₂.

In some embodiments of a method or biphasic mixture provided herein involving a temperature below 24° C., the temperature is between 0-24° C. Optionally, the temperature is between 0-23° C. Optionally, the temperature is between 1-20° C. Optionally, the temperature is between 1-15° C. Optionally, the temperature is between 1-10° C.

In some embodiments of a method provided herein involving a sample ready for endotoxin testing, the sample ready for endotoxin testing further is tested for endotoxin via a Limulus Amebocyte Lysate (LAL) assay. Optionally, the LAL assay is a gel-clot LAL assay, a chromogenic LAL assay, a turbidimetric LAL assay, or a LAL assay comprising recombinant Factor C.

In some embodiments of a method provided herein involving a sample ready for endotoxin testing, a greater amount of endotoxin can be detected in the sample ready for endotoxin testing than from an otherwise identical corresponding solution containing the molecule of interest and the masked endotoxin that was not subject to a method provided herein.

In some embodiments of a biphasic mixture or method provided herein, the biphasic mixture, solution comprising a molecule of interest, Liquid A, or Liquid B does not contain bovine serum albumin (BSA).

In some embodiments of a biphasic mixture or method provided herein comprising an antibody, the antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

In some embodiments of a biphasic mixture or method provided herein comprising an antibody, the antibody comprises a heavy chain variable region (VH) having the sequence shown in SEQ ID NO: 1. In some embodiments, the antibody comprises a light chain variable region (VL) having the amino acid sequence of SEQ ID NO: 2. In some embodiments, the antibody comprises a heavy chain variable region (VH) having the sequence shown in SEQ ID NO: 1 and a light chain variable region (VL) having the amino acid sequence of SEQ ID NO: 2.

In some embodiments of a biphasic mixture or method provided herein comprising an antibody, the antibody comprises a heavy chain having the amino acid sequence shown in SEQ ID NO: 9 and a light chain having the amino acid sequence shown in SEQ ID NO: 10. In some embodiments, the C-terminal lysine (K) of the heavy chain amino acid sequence of SEQ ID NO: 9 is optional.

In some embodiments, a biphasic mixture or method as provided herein involving 1-dodecanol may alternatively contain or be performed with 2-dodecanol (IUPAC name: dodecan-2-ol; CAS ID: 10203-28-8).

In some embodiments, a biphasic mixture or method as provided herein involving a sample (e.g. drug product) spiked with endotoxin may be spiked with 1000 Endotoxin Unit (EU)/ml Control Standard Endotoxin (CSE). In some embodiments, the sample is spiked with 50-2000, 80-1250, or 500-1500 EU CSE/ml. In some embodiments, the sample is spiked with about 50, 80, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 EU CSE/ml.

### DETAILED DESCRIPTION

Provided herein are methods and compositions for unmasking endotoxin and overcoming situations of Low Endotoxin Recovery (LER) related to endotoxin detection assays.

Methods provided herein may be used to prepare a sample comprising a molecule of interest for endotoxin testing. In some embodiments, methods provided herein are useful for situations where a sample to be tested for endotoxin has been identified as exhibiting Low Endotoxin Recovery/endotoxin masking. In these situations, it has been identified that endotoxin which may be present in the sample cannot be readily be detected by endotoxin detection assays. In these situations, it is desirable to “unmask” endotoxin which may be present in the sample, in order to accurately detect the presence and/or amount of endotoxin in the sample.

In addition, methods provided herein may also be used in any situation where it desirable to unmask any endotoxin that may be present in a sample of interest. In other words, it is not necessary to know that a sample contains masked endotoxin and/or exhibits LER to use a method provided herein; methods provided herein are useful to prepare any sample for endotoxin testing.

Methods of unmasking endotoxin/overcoming LER as provided herein may be used to treat a sample comprising a molecule of interest prior to an endotoxin detection assay.

Endotoxin detection assays are known in the art. In some embodiments, the endotoxin detection assay is a Limulus amoebocyte lysate (LAL) assay. LAL assays may be performed via various different methodologies, such as chromogenic, turbidimetric, or gel clot. LAL assays may be performed using recombinant proteins (e.g. recombinant Factor C). LAL assay reagents and kits are available commercially, such as from the companies Lonza, InvivoGen, Pierce, GenScript, and Charles River.

In some embodiments, methods and compositions provided herein include 1-dodecanol. 1-dodecanol is 12 carbon fatty alcohol. 1-dodecanol is also known as lauryl alcohol, dodecanol, and dodecyl alcohol. The IUPAC name is dodecan-1-ol, and the CAS number is 112-53-8. The term “1-dodecanol” is used interchangeably herein with the term “dodecanol”. Unless otherwise indicated, as used herein, the terms “1-dodecanol”, “dodecanol”, etc. refer to highly purified 1-dodecanol (i.e. ≥98% pure). 1-dodecanol is a solid at ambient/room temperature; it has a melting point of 24° C./75° F. References herein to “liquid phase 1-dodecanol”, “molten 1-dodecanol”, and the like refer to highly purified 1-dodecanol (i.e. ≥98% pure) that is in the liquid phase due to being at a temperature above its melting point (i.e. above 24° C./75° F.). Thus, as used herein, “liquid phase 1-dodecanol” and the like does not refer 1-dodecanol that has been solubilized in a solvent (e.g. ethanol).

In some embodiments, methods and compositions provided herein for unmasking endotoxin/overcoming LER involve the liquid phase to solid phase change of 1-dodecanol, or vice-versa. In some embodiments, methods and compositions provided herein involve adding liquid phase 1-dodecanol to a sample comprising a molecule of interest and endotoxin, followed by cooling the sample to solidify the 1-dodecanol, as an aspect of the process for unmasking endotoxin/overcoming LER. Similarly, compositions comprising a molecule of interest and endotoxin and liquid phase or solid phase 1-dodecanol are also provided herein.

1-dodecanol is less dense than water. For example, 1-dodecanol has a density of approximately 831 kg/m³. In contrast, water has a density of approximately 997 kg/m³. Accordingly, when liquid 1-dodecanol and water (or an aqueous solution) are mixed, the 1-dodecanol settles in a layer above the water (i.e. the 1-dodecanol floats on the water).

In some embodiments, methods and compositions provided herein include PYROSPERSE™. PYROSPERSE™ is a dispersing agent commercially available from Lonza (catalog number N188). Per Lonza product information, PYROSPERSE™ can help eliminate endotoxin binding or masking in some samples. Per Lonza product information, PYROSPERSE™ is a metallo-modified polyanionic dispersant.

In some embodiments, methods and compositions provided herein comprise solutions containing Ca²⁺ and/or Mg²⁺ ions. Solutions containing Ca²⁺ and/or Mg²⁺ ions may be prepared by methods known to persons of skill in the art. For example, solutions containing Ca²⁺ ions can be prepared by dissolving CaCl₂ in water, and solutions containing Mg²⁺ ions can be prepared by dissolving MgCl₂ in water. In some embodiments, solutions containing Ca²⁺ and/or Mg²⁺ ions provided herein can contain 0.1-2 M Ca²⁺ and/or Mg²⁺ ions. Optionally, a solution containing Ca²⁺ and/or Mg²⁺ ions provided herein can contain 0.1, 0.2, 0.3, 0.4, 0.5, 1, 1.5, or 2 M Ca²⁺ and/or Mg²⁺ ions. Optionally, a solution containing Ca²⁺ and/or Mg²⁺ ions provided herein may contain only Ca²⁺ ions or only Mg²⁺ ions. Optionally, a solution containing Ca²⁺ and/or Mg²⁺ ions provided herein may contain a mixture of Ca²⁺ ions and Mg²⁺ ions, wherein the combined combination of Ca²⁺ ions and Mg²⁺ ions is 0.1, 0.2, 0.3, 0.4, 0.5, 1, 1.5, or 2 M cations having a 2+ charge.

In any embodiment provided herein involving a ratio involving a volume (e.g. a ratio of microliters of 1-dodecanol to microliters of solution comprising the molecule of interest and masked endotoxin), the volumes provided in the ratio are exemplary, and may be increased or decreased according to the recited ratio. For example, when the above ratio is described as being “between a) 0.2 microliters 1-dodecanol per 10 microliters solution and b) 2 microliters 1-dodecanol per 10 microliters solution”, this also encompasses multiples of each of these numbers, when the multiple is the same for each number. Thus, the ratio also encompasses, for example, 5× multiples of the phrase: “between a) 1 microliters 1-dodecanol per 50 microliters solution and b) 10 microliters 1-dodecanol per 50 microliters solution”, 10× multiples of the phrase: “between a) 2 microliters 1-dodecanol per 100 microliters solution and b) 20 microliters 1-dodecanol per 100 microliters solution”, 20× multiples of the phrase : “between a) 4 microliters 1-dodecanol per 200 microliters solution and b) 40 microliters 1-dodecanol per 200 microliters solution”, etc.

Methods and compositions provided herein may be used for unmasking endotoxins in a solution comprising a molecule of interest and masked endotoxin.

In some embodiments, the molecule of interest is a pharmaceutical product. In some embodiments, the molecule of interest is a protein. In some embodiments, the molecule of interest is an antibody.

In some embodiments, the molecule of interest is an anti-nerve growth factor (NGF) antibody. In one aspect, the anti-NGF antibody binds to NGF and inhibits binding of NGF to trkA and/or p75.

In some embodiments, the molecule of interest is an antibody, and the antibody comprises three CDRs from the heavy chain variable region of SEQ ID NO: 1. In some embodiments, the antibody comprises three CDRs from the light chain variable region of SEQ ID NO: 2. In some embodiments the antibody comprises three CDRs from the heavy chain variable region of SEQ ID NO: 1 and three CDRs from the light chain variable region of SEQ ID NO: 2. In some embodiments, the CDRs may be defined in accordance with any of Kabat, Chothia, extended, AbM, contact, and/or conformational definitions. In some embodiments, the CDRS shown in SEQ ID NO:3, SEQ ID NO:4, SEQ ID NO:5, SEQ ID NO:6, SEQ ID NO:7, and SEQ ID NO:8 are determined by a combination of the Kabat and Chothia methods.

Exemplary antibody sequences of molecules of interest provided herein include, but are not limited to, the sequences listed below.

TABLE 1

| SEQ | |
|---|---|
| ID | |
| NO: | Sequence |
| 1 | Variable heavy chain region: |
| | QVQLQESGPGLVKPSETLSLTCTVSGFSLI |
| | GYDLNWIRQPPGKGLEWIGIIWGDGTTDYN |
| | SAVKSRVTISKDTSKNQFSLKLSSVTAADT |
| | AVYYCARGGYWYATSYYFDYWGQGTLVTVS |
| 2 | Variable light chain region: |
| | DIQMTQSPSSLSASVGDRVTITCRASQSIS |
| | NNLNWYQQKPGKAPKLLIYYTSRFHSGVPS |
| | RFSGSGSGTDFTFTISSLQPEDIATYYCQQ |
| | EHTLPYTFGQGTKLEIKRT |
| 3 | Extended HCDR1: |
| | GFSLIGYDLN |
| 4 | Extended HCDR2: |
| | IIWGDGTTDYNSAVKS |
| 5 | Extended HCDR3: |
| | GGYWYATSYYFDY |
| 6 | Extended LCDR1: |
| | RASQSISNNLN |
| 7 | Extended LCDR2: |
| | YTSRFHS |
| 8 | Extended LCDR3: |
| | QQEHTLPYT |
| 9 | Heavy chain*: |
| | QVQLQESGPGLVKPSETLSLTCTVSGFSLI |
| | GYDLNWIRQPPGKGLEWIGIIWGDGTTDYN |
| | SAVKSRVTISKDTSKNQFSLKLSSVTAADT |
| | AVYYCARGGYWYATSYYFDYWGQGTLVTVS |
| | SASTKGPSVFPLAPCSRSTSESTAALGCLV |
| | KDYFPEPVTVSWNSGALTSGVHTFPAVLQS |
| | SGLYSLSSVVTVPSSNFGTQTYTCNVDHKP |
| | SNTKVDKTVERKCCVECPPCPAPPVAGPSV |
| | FLFPPKPKDTLMISRTPEVTCVVVDVSHED |
| | PEVQFNWYVDGVEVHNAKTKPREEQFNSTF |
| | RVVSVLTVVHQDWLNGKEYKCKVSNKGLPS |
| | SIEKTISKTKGQPREPQVYTLPPSREEMTK |
| | NQVSLTCLVKGFYPSDIAVEWESNGQPENN |
| | YKTTPPMLDSDGSFFLYSKLTVDKSRWQQG |
| | NVFSCSVMHEALHNHYTQKSLSLSPGK |
| 10 | Light chain: |
| | DIQMTQSPSSLSASVGDRVTITCRASQSIS |
| | NNLNWYQQKPGKAPKLLIYYTSRFHSGVPS |
| | RFSGSGSGTDFTFTISSLQPEDIATYYCQQ |
| | EHTLPYTFGQGTKLEIKRTVAAPSVFIFPP |
| | SDEQLKSGTASVVCLLNNFYPREAKVQWKV |
| | DNALQSGNSQESVTEQDSKDSTYSLSSTLT |
| | LSKADYEKHKVYACEVTHQGLSSPVTKSFN |
| | RGEC |

[*C-terminal lysine (K) of the heavy chain amino acid sequence of SEQ ID NO: 9 is optional]

In one embodiment, a molecule of interest for a method provided herein is the antibody tanezumab. The antibody “tanezumab” is a humanized immunoglobulin G Type 2 (IgG2) monoclonal antibody directed against human nerve growth factor (NGF). Tanezumab binds to human NGF with high affinity and specificity and blocks the activity of NGF effectively in cell culture models. Tanezumab and/or its murine precursor have been shown to be an effective analgesic in animal models of pathological pain including arthritis, cancer pain, and post-surgical pain. Tanezumab has the sequences for the variable heavy chain region and variable light chain region of SEQ ID Nos: 1 and 2, respectively. The heavy chain and light chain sequences are provided in SEQ ID NO: 9 and 10, respectively, wherein the C-terminal lysine (K) of the heavy chain amino acid sequence of SEQ ID NO: 9 is optional. Sequences of tanezumab are provided in Table 1 above. Tanezumab is described, as antibody E3, in WO2004/058184, herein incorporated by reference.

In some embodiments the anti-NGF antibody, such as tanezumab, is in a drug product formulation, such as described in WO2010/032220, herein incorporated by reference.

In some embodiments, the formulation is a liquid formulation and comprises an anti-NGF antibody at a concentration of about 2.5 mg/ml, 5 mg/ml, 10 mg/ml or 20 mg/ml; and a histidine buffer.

In some embodiments, the formulation further comprises a surfactant which may be polysorbate 20. In some embodiments, the formulation further comprises trehalose dehydrate or sucrose. In some embodiments, the formulation further comprises a chelating agent, which may be EDTA; in some embodiments disodium EDTA. In some embodiments, the formulation is of pH 6.0±0.3.

In some embodiments, the formulation comprises about 2.5 mg/ml, 5 mg/ml, 10 mg/ml or 20 mg/ml tanezumab; about 10 mM histidine buffer; about 84 mg/ml trehalose dehydrate; about 0.1 mg/ml Polysorbate 20; about 0.05 mg/ml disodium EDTA; wherein the formulation is of a pH 6.0±0.3.

In some embodiments the formulation comprises about 2.5 mg/ml or 5 mg/ml tanezumab. In some embodiments, the formulation has a total volume of about 1 ml.

In some embodiments the formulation is contained in a glass or plastic vial or syringe. In some embodiments the formulation is contained in a pre-filled glass or plastic vial or syringe.

Incorporated by reference herein for all purposes is the content of U.S. Provisional Patent Application Nos. 62/951,496 (filed Dec. 20, 2019) and 63/117,201 (Filed Nov. 23, 2020).

Additional exemplary embodiments provided herein included the embodiments (E) as provided below:

E1. A method of unmasking endotoxin in a solution comprising a molecule of interest and masked endotoxin, the method comprising: a) adding liquid phase 1-dodecanol to a solution containing the molecule of interest and the masked endotoxin; and b) cooling the liquid phase 1-dodecanol and solution containing the molecule of interest to a temperature below 24° C., such that the 1-dodecanol solidifies and there is a remaining liquid portion and the solidified 1-dodecanol.
E2. The method of E1, wherein the remaining liquid portion is “Liquid A”, and wherein the method further comprises adding PYROSPERSE™ solution or a solution comprising CaCl₂ to Liquid A, to generate a mixture (“Liquid B”) comprising PYROSPERSE™ or CaCl₂ and Liquid A.
E3. The method of E2, further comprising incubating Liquid B.
E4. The method of E3, further comprising, after incubating Liquid B, vortexing Liquid B.
E5. The method of any one of E2-E4, further comprising diluting a portion of Liquid B into a buffer comprising MgSO₄ to yield a sample ready for endotoxin testing, wherein the previously masked endotoxin is unmasked in the sample ready for endotoxin testing.
E6. A method of unmasking endotoxin in a solution comprising a molecule of interest and masked endotoxin, the method comprising: a) adding liquid phase 1-dodecanol to a solution containing the molecule of interest and the masked endotoxin; b) cooling the liquid phase 1-dodecanol and solution containing the molecule of interest to a temperature below 24° C., such that the 1-dodecanol solidifies and there is a remaining liquid portion (“Liquid A”) and the solid 1-dodecanol; c) adding PYROSPERSE™ solution or a solution comprising CaCl₂ to Liquid A, to generate a mixture (“Liquid B”) comprising PYROSPERSE™ or a solution comprising CaCl₂ and Liquid A; d) incubating Liquid B; e) vortexing Liquid B; f) diluting a portion of Liquid B into a buffer comprising MgSO₄ to yield a sample ready for endotoxin testing; wherein the previously masked endotoxin is unmasked in the sample of step f).
E7. The method of any one of E1-E6, wherein the molecule of interest is a protein.
E8. The method of E7, wherein the protein is an antibody.
E9. The method of E8, wherein the antibody is tanezumab.
E10. The method of any one of E1-E9, wherein the solution comprising a molecule of interest is a tanezumab drug product.
E11. The method of any one of E9-E10, wherein the solution comprises 2.5 mg/ml, 5 mg/ml, 10 mg/ml, or 20 mg/ml tanezumab.
E12. The method of any one of E1-E11, wherein the 1-dodecanol is ≥98% pure 1-dodecanol.
E13. The method of any one of E1-E12, wherein the ratio of microliters of 1-dodecanol to microliters of solution comprising the molecule of interest and masked endotoxin is between a) 0.2 microliters 1-dodecanol per 10 microliters solution and b) 2 microliters 1-dodecanol per 10 microliters solution.
E14. The method of E13, wherein the ratio of microliters of 1-dodecanol to microliters of solution comprising the molecule of interest and masked endotoxin is between a) 0.5 microliters 1-dodecanol per 10 microliters solution and b) 1.5 microliters 1-dodecanol per 10 microliters solution.
E15. The method of any one of E1-E14, wherein the ratio of microliters of 1-dodecanol to microliters of solution comprising the molecule of interest and masked endotoxin is 1 microliter 1-dodecanol per 9 microliters solution.
E16. The method of any one of E1-E15, wherein the liquid phase 1-dodecanol and solution containing the molecule of interest are cooled in an ice water bath or cooling block for 0.5-20 minutes.
E17. The method of any one of E1-E16, wherein the liquid phase 1-dodecanol and solution containing the molecule of interest are cooled for 1, 2, or 3 minutes.
E18. The method of any one of E2-E17, wherein the solution comprising CaCl₂ comprises a concentration of CaCl₂ of 1 M, 2M, or a value between 1M and 2M.
E19. The method of E16, wherein the solution comprising CaCl₂ comprises a concentration of CaCl₂ of 1.5 M.
E20. The method of any one of E2-E19, wherein the ratio of microliters of PYROSPERSE™ or 1.5M CaCl₂ solution to Liquid A is between a) 0.5 microliters PYROSPERSE™ or 1.5M CaCl₂ solution per 100 microliters Liquid A and b) 5 microliters PYROSPERSE™ or 1.5M CaCl₂ solution per 100 microliters Liquid A.
E21. The method of any one of E2-E20, wherein the ratio of microliters of PYROSPERSE™ or 1.5M CaCl₂ solution to Liquid A is 3 microliters PYROSPERSE™ or 1.5M CaCl₂ solution per 100 microliters Liquid A.
E22. The method of any one of E3-E21, wherein Liquid B is incubated at ambient temperature.
E23. The method of any one of E3-E22, wherein Liquid B is incubated for 0.5-20 minutes.
E24. The method of any one of E3-E23, wherein Liquid B is incubated for 8-10 minutes.
E25. The method of any one of E4-E24, wherein Liquid B is vortexed for 0.1-5 minutes.
E26. The method of any one of E4-E25, wherein Liquid B is vortexed for 1 minute.
E27. The method of any one of E5-E26, wherein the buffer comprising MgSO₄ comprises 1-50 mM MgSO₄.
E28. The method of any one of E5-E27, wherein the buffer comprising MgSO₄ comprises 10 mM MgSO₄.
E29. The method of any one of E5-E28, wherein the buffer comprising MgSO₄ further comprises 2-100 mM Tris, pH 6.8-7.6.
E30. The method of any one of E5-E29, wherein the buffer comprising MgSO₄ further comprises 20 mM Tris, pH 6.8-7.6.
E31. The method of any one of E5-E30, wherein the portion of Liquid B is diluted into the buffer comprising MgSO₄ at a ratio between a) 1 microliter Liquid B per 100 microliters MgSO₄ buffer and b) 1 microliter Liquid B per 3000 microliters MgSO₄ buffer.
E32. The method of any one of E5-E31, wherein the portion of Liquid B is diluted into the buffer comprising MgSO₄ at a ratio of 1 microliter Liquid B per 2000 microliters MgSO₄ buffer.
E33. A method of unmasking endotoxin in a solution comprising a molecule of interest and masked endotoxin, the method comprising: a) adding 100 microliters liquid phase 1-dodecanol to 900 microliters of a solution containing the molecule of interest and the masked endotoxin; b) cooling the liquid phase 1-dodecanol and solution containing the molecule of interest in an ice water bath or cooling block for 2 minutes to a temperature below 24° C., such that the 1-dodecanol solidifies and there is a remaining liquid portion (“Liquid A”) and the solid 1-dodecanol; c) adding 30 microliters PYROSPERSE™ solution or 30 microliters of a solution comprising 1.5 M CaCl₂ to Liquid A, to generate a mixture (“Liquid B”) comprising PYROSPERSE™ or a solution comprising CaCl₂ and Liquid A; d) incubating Liquid B at ambient temperature for 8-10 minutes; e) after incubating Liquid B, vortexing Liquid B for 1 minute; f) diluting a portion of Liquid B from step e) into a buffer comprising 10 mM MgSO₄ for a dilution of 1:2000, to yield a sample ready for endotoxin testing; wherein the previously masked endotoxin is unmasked in the sample of step f).
E34. The method of any one of E5-E33, wherein the sample ready for endotoxin testing further is tested for endotoxin via a Limulus Amebocyte Lysate (LAL) assay.
E35. The method of E34, wherein the LAL assay is a gel-clot LAL assay, a chromogenic LAL assay, a turbidimetric LAL assay, or a LAL assay comprising recombinant Factor C.
E36. The method of any one of E5-E35, wherein a greater amount of endotoxin can be detected in the sample ready for endotoxin testing than from an otherwise identical corresponding solution containing the molecule of interest and the masked endotoxin that was not subject to the method of any one of E5-E35.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995), as well as in subsequent editions and corresponding websites of the above references, as applicable.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

### EXAMPLES

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1: Identification of Low Endotoxin Recovery (LER) in a Tanezumab Drug Product

This example describes the identification of Low Endotoxin Recovery (LER) in a tanezumab drug product.

On Day 0, tanezumab drug product (DP) containing 2.5 mg/ml, 5 mg/ml, 10 mg/ml, or 20 mg/ml tanezumab was spiked with 1000 Endotoxin Unit (EU)/ml Control Standard Endotoxin (CSE). The endotoxin-spiked tanezumab DP was incubated for 14 days at ambient temperature.

Samples of endotoxin-spiked tanezumab DP were taken on days 0, 4, 9, and 14, and assayed for endotoxin via standard kinetic chromogenic (KCA) Limulus amebocyte lysate (LAL) method (per USP <85>, Ph. Eur. 2.6.14, and JP 4.01 standards). Endotoxin recovery from the spiked tanezumab DP on days 4, 9, and 14 was below the acceptable range (50-200%); i.e. less than 50% of the spiked endotoxin was recovered on each of days 4, 9, and 14. Specifically, around 0% of the spiked endotoxin was recovered on days 4, 9, and 14. On Day 0, the % endotoxin recovery was within the acceptable range (around 70%).

Since endotoxin recovery from the spiked tanezumab DP was below the acceptable range on each of days 4, 9, and 14, this indicated LER in the tanezumab drug product.

### Example 2: Development of a Method to Overcome LER in Tanezumab Drug Product

This example describes the development of a new method to overcome Low Endotoxin Recover (LER) in the tanezumab drug product (DP).

After the finding of LER in tanezumab DP as described in Example 1, multiple different known methods for addressing LER/unmasking endotoxins were attempted. However, these efforts did not identify an effective method for overcoming LER in the tanezumab DP.

Next, a wide range of experimental conditions were tested in an effort to develop an effective method for overcoming LER in the tanezumab DP. As result of this work, the surprising combination of reagents and conditions described below was identified as being effective for overcoming LER and unmasking endotoxins in the tanezumab DP. The development of this method is useful, as it provides a method of pre-treating a tanezumab DP prior to endotoxin testing, so that an endotoxin detection assay can be accurately performed on the tanezumab DP. As described in Example 1, if tanezumab DP is not pre-treated with an effective method for unmasking endotoxins prior to the endotoxin detection assay, endotoxins in the tanezumab DP will be “masked’ and not effectively detected (i.e. LER will occur), potentially leading to inaccurate results from the endotoxin detection assay.

The starting material for this method was endotoxin-spiked tanezumab DP (2.5 mg/ml, 5 mg/ml, 10 mg/ml, or 20 mg/ml) that contained 1000 Endotoxin Unit (EU)/ml Control Standard Endotoxin (CSE), and that had been incubated for 4, 9, or 14 days at ambient temperature (“starting material”).

Liquid phase 1-dodecanol (≥98% pure) was used for this assay. 1-Dodecanol is solid at room temperature (melting point 24° C.); accordingly, 1-dodecanol was warmed to a temperature around 30-35° C. (e.g. 32° C.) to liquid phase for use in this assay.

900 microliters of the starting material was transferred to a glass tube. 100 microliters of 1-dodecanol in liquid phase at ˜32° C. was slowly added to the starting material via gentle delivery to minimize mixing between the starting material and 1-dodecanol. Upon addition of the 1-dodecanol to the starting material, the glass tube was immediately put in an ice water bath for 2 minutes. After the 2 minutes, the 1-dodecanol had returned to a solid state, and formed a compact solid in the tube. Accordingly, at this point, there was a liquid portion (“Liquid A”; approximately 900 microliters volume) and a solid portion in the tube.

The tube was removed from the ice bath, and 30 microliters of dispersant [PYROSPERSE™ (Lonza)] was added to Liquid A, resulting a mixture of Liquid A+PYROSPERSE™ (together: “Liquid B”; approximate volume: 930 microliters). The tube was then left undisturbed for 10 minutes at ambient temperature.

After the 10 minute ambient temperature incubation, the tube was vortexed for 1 minute. A small volume of the liquid phase from the tube (Liquid B) was removed, and diluted in buffer containing 10 mM MgSO4, 20 mM Tris pH 6.8-7.6, to yield a 1/2000 dilution of Liquid B in the buffer. For example, 20 microliters of Liquid B was added to 980 microliters of buffer and mixed, to yield 1 mL of a 1:50 dilution of Liquid B. Then, 25 microliters of the 1:50 dilution of Liquid B was added to 975 microliters of buffer, to yield 1 mL of a 1:2000 dilution of Liquid B. (Other suitable steps to achieve a 1:2000 dilution were also used.)

The 1:2000 dilution of Liquid B was then vortexed, and tested for endotoxin according to a standard KCA LAL method (per USP <85>, Ph. Eur. 2.6.14, and JP 4.01 standards).

Treatment of tanezumab DP according the method described above consistently overcame the LER observed in tanezumab DP. In other words, endotoxin recovery from the spiked tanezumab DP on days 4, 9, and 14 was consistently within the acceptable range (50-200%) when the spiked tanezumab DP is treated with the above method to overcome LER/unmask endotoxins before testing the sample for endotoxin.

### Example 3: Further Development of the Method to Overcome LER in Tanezumab Drug Product

This example describes the further development of a new method to overcome Low Endotoxin Recover (LER) in the tanezumab drug product (DP).

The method as described in Example 2 was repeated multiple times, while varying one or more of the parameters as compared to the method of Example 2.

After extensive testing the following conditions as provided in Table 2, right column, were determined to also be acceptable variations of the method as described in Example 2 to overcome LER in tanezumab DP.

TABLE 2

| | Corresponding Acceptable |
|---|---|
| Action in Example 2 | Alternatives |
| Tanezumab DP (2.5-20 | Tanezumab DP (2.5-20 |
| mg/ml) spiked with 1000 | mg/ml) spiked with 80- |
| Endotoxin Unit (EU) Control | 1250 EU CSE/ml |
| Standard Endotoxin (CSE)/ml | |
| Preparation of a 10% (v/v) | 5%-11% 1-dodecanol/ |
| 1-dodecanol/tanezumab DP | tanezumab DP mixture |
| mixture (e.g. 100 μl liquid | |
| phase 1-dodecanol + | |
| 900 μl tanezumab DP) | |
| Preparation of an | 0.5%-4% (v/v) dispersant/ |
| approximately 3% (v/v) | Liquid A mixture (together |
| dispersant (e.g. | referred to as “Liquid B”); |
| PYROSPERSE ™)/ | Alternatively, another option |
| Liquid A mixture (together | is no dispersant (i.e. 0 μl |
| referred to as “Liquid B”) | dispersant solution). |
| [e.g. 30 μl dispersant | |
| solution + ~900 μl | |
| Liquid A = Liquid B (contains | |
| approximately 3% v/v | |
| dispersant/Liquid A)], | |
| Incubating Liquid B at ambient | Incubation time <1 min- |
| temperature for 8-10 minutes | incubation time >10 minutes |
| | Alternatively, another option |
| | is no incubation time (0 |
| | minutes) (The incubation step |
| | improves consistency but is |
| | optional.) |
| Vortexing Liquid B for | Vortexing Liquid B for |
| 1 minute | 30-60 seconds |
| Diluting a portion of vortexed | Diluent buffers: 1-10 mM |
| Liquid B into a buffer containing | MgSO₄; 2-20 mM |
| 10 mM MgSO₄, for a dilution of | Tris pH 6.8-7.6 |
| 1:2000, to yield a sample ready | Dilution factors: |
| for endotoxin testing | 1:100-1:2500 |

Treatment of tanezumab DP according the method of Example 2, with the variations described above in Table 2, right column, consistently overcame the LER observed in tanezumab DP. In addition, it is further noted that Table 2 does not include all possible acceptable alternative values for the method as described in Example 2; rather it provides alterative values that have been experimentally confirmed. Based on the teachings provided herein, a person of skill in the art would understand that additional alternatives are within the scope of the invention provided herein.

Table 3 below provides experimental details for multiple successful variants of the method of Example 2 (variant #s 1-48). In Table 3, each horizontal row depicts details of a successful variant of the method of Example 2. A variant method was considered successful if, after treatment with the variant method, the endotoxin recovery from the spiked tanezumab DP on day 2-14 was within the acceptable range (50-200%). [LER is observed already on day 1; accordingly, samples were tested on any one of days 2-14 to assess the efficacy of a method provided in Table 3 to overcome LER.] The headings of the vertical columns of Table 3 list various aspects of the method of Example 2, and the respective boxes provide the relevant value of the respective aspect for each variant method. For each of the variants methods in Table 3, 900 μl, 950 μl, or 1000 μl DP was used. The dispersant in the methods of Table 3 was PYROSPERSE™. As shown in Table 3, some of the variant methods do not include addition of a dispersant (#s 24, 38, and 48). Accordingly, addition of a dispersant is optional with methods provided herein.

TABLE 3

| | | | 1- | | | | |
|---|---|---|---|---|---|---|---|
| | DP | Endotoxin | Dodecanol | Dispersant | | | % |
| Variant | Conc | Spike | volume | volume | | Dilution | Endotoxin |
| # | (mg/ml) | (EU/ml) | (μl) | (μl) | Diluent | Factor | Recovery |
| 1 | 2.5 | 80 | 50 | 10 | 10mM | 100 | 64 |
| | | | | | MgSO4/ | | |
| | | | | | 20 mM | | |
| | | | | | Tris | | |
| 2 | 2.5 | 80 | 100 | 30 | 10 mM | 100 | 68 |
| | | | | | MgSO4/ | | |
| | | | | | 20 mM | | |
| | | | | | Tris | | |
| 3 | 2.5 | 80 | 50 | 10 | 1 mM | 100 | 102 |
| | | | | | MgSO4/ | | |
| | | | | | 2 mM Tris | | |
| 4 | 2.5 | 80 | 100 | 30 | 1 mM | 100 | 58 |
| | | | | | MgSO4/ | | |
| | | | | | 2 mM Tris | | |
| 5 | 2.5 | 80 | 50 | 10 | 5 mM | 100 | 53 |
| | | | | | MgSO4/ | | |
| | | | | | 10 mM | | |
| | | | | | Tris | | |
| 6 | 2.5 | 80 | 100 | 30 | 5 mM | 100 | 81 |
| | | | | | MgSO4/ | | |
| | | | | | 10 mM | | |
| | | | | | Tris | | |
| 7 | 2.5 | 80 | 100 | 30 | 10 mM | 295 | 78 |
| | | | | | MgSO4/ | | |
| | | | | | 20 mM | | |
| | | | | | Tris | | |
| 8 | 2.5 | 200 | 100 | 30 | 10 mM | 295 | 87 |
| | | | | | MgSO4/ | | |
| | | | | | 20 mM | | |
| | | | | | Tris | | |
| 9 | 2.5 | 1000 | 50 | 5 | 10 mM | 100 | 59 |
| | | | | | MgSO4/ | | |
| | | | | | 20 mM | | |
| | | | | | Tris | | |
| 10 | 2.5 | 1000 | 50 | 10 | 10 mM | 100 | 78 |
| | | | | | MgSO4/ | | |
| | | | | | 20 mM | | |
| | | | | | Tris | | |
| 11 | 2.5 | 1000 | 50 | 20 | 10 mM | 100 | 63 |
| | | | | | MgSO4/ | | |
| | | | | | 20 mM | | |
| | | | | | Tris | | |
| 12 | 2.5 | 1000 | 50 | 30 | 10 mM | 100 | 115 |
| | | | | | MgSO4/ | | |
| | | | | | 20 mM | | |
| | | | | | Tris | | |
| 13 | 2.5 | 1000 | 100 | 30 | 10 mM | 100 | 106 |
| | | | | | MgSO4/ | | |
| | | | | | 20 mM | | |
| | | | | | Tris | | |
| 14 | 10 | 1000 | 50 | 20 | 10 mM | 100 | 65 |
| | | | | | MgSO4/ | | |
| | | | | | 20 mM | | |
| | | | | | Tris | | |
| 15 | 10 | 1000 | 100 | 30 | 10 mM | 100 | 77 |
| | | | | | MgSO4/ | | |
| | | | | | 20 mM | | |
| | | | | | Tris | | |
| 16 | 20 | 1000 | 50 | 10 | 10 mM | 100 | 57 |
| | | | | | MgSO4/ | | |
| | | | | | 20 mM | | |
| | | | | | Tris | | |
| 17 | 20 | 1000 | 50 | 30 | 10 mM | 100 | 91 |
| | | | | | MgSO4/ | | |
| | | | | | 20 mM | | |
| | | | | | Tris | | |
| 18 | 20 | 1000 | 100 | 10 | 10 mM | 100 | 100 |
| | | | | | MgSO4/ | | |
| | | | | | 20 mM | | |
| | | | | | Tris | | |
| 19 | 20 | 1000 | 100 | 30 | 10 mM | 100 | 113 |
| | | | | | MgSO4/ | | |
| | | | | | 20 mM | | |
| | | | | | Tris | | |
| 20 | 2.5 | 1000 | 50 | 5 | 1 mM | 100 | 56 |
| | | | | | MgSO4/ | | |
| | | | | | 2 mM Tris | | |
| 21 | 2.5 | 1000 | 50 | 10 | 1 mM | 100 | 68 |
| | | | | | MgSO4/ | | |
| | | | | | 2 mM Tris | | |
| 22 | 2.5 | 1000 | 50 | 20 | 1 mM | 100 | 60 |
| | | | | | MgSO4/ | | |
| | | | | | 2 mM Tris | | |
| 23 | 2.5 | 1000 | 50 | 30 | 1 mM | 100 | 131 |
| | | | | | MgSO4/ | | |
| | | | | | 2 mM Tris | | |
| 24 | 2.5 | 1000 | 50 | 0 | 1 mM | 100 | 60 |
| | | | | | MgSO4/ | | |
| | | | | | 2 mM Tris | | |
| 25 | 2.5 | 1000 | 100 | 5 | 1 mM | 100 | 51 |
| | | | | | MgSO4/ | | |
| | | | | | 2 mM Tris | | |
| 26 | 2.5 | 1000 | 100 | 10 | 1 mM | 100 | 120 |
| | | | | | MgSO4/ | | |
| | | | | | 2 mM Tris | | |
| 27 | 2.5 | 1000 | 100 | 30 | 1 mM | 100 | 107 |
| | | | | | MgSO4/ | | |
| | | | | | 2 mM Tris | | |
| 28 | 10 | 1000 | 50 | 20 | 1 mM | 100 | 92 |
| | | | | | MgSO4/ | | |
| | | | | | 2 mM Tris | | |
| 29 | 10 | 1000 | 100 | 30 | 1 mM | 100 | 172 |
| | | | | | MgSO4/ | | |
| | | | | | 2 mM Tris | | |
| 30 | 20 | 1000 | 50 | 5 | 1 mM | 100 | 52 |
| | | | | | MgSO4/ | | |
| | | | | | 2 mM Tris | | |
| 31 | 20 | 1000 | 50 | 10 | 1 mM | 100 | 58 |
| | | | | | MgSO4/ | | |
| | | | | | 2 mM Tris | | |
| 32 | 20 | 1000 | 50 | 30 | 1 mM | 100 | 89 |
| | | | | | MgSO4/ | | |
| | | | | | 2 mM Tris | | |
| 33 | 20 | 1000 | 100 | 30 | 1 mM | 100 | 126 |
| | | | | | MgSO4/ | | |
| | | | | | 2 mM Tris | | |
| 34 | 2.5 | 1000 | 50 | 5 | 5 mM | 100 | 96 |
| | | | | | MgSO4/ | | |
| | | | | | 10 mM | | |
| | | | | | Tris | | |
| 35 | 2.5 | 1000 | 50 | 10 | 5 mM | 100 | 88 |
| | | | | | MgSO4/ | | |
| | | | | | 10 mM | | |
| | | | | | Tris | | |
| 36 | 2.5 | 1000 | 50 | 20 | 5 mM | 100 | 62 |
| | | | | | MgSO4/ | | |
| | | | | | 10 mM | | |
| | | | | | Tris | | |
| 37 | 2.5 | 1000 | 50 | 30 | 5 mM | 100 | 135 |
| | | | | | MgSO4/ | | |
| | | | | | 10 mM | | |
| | | | | | Tris | | |
| 38 | 2.5 | 1000 | 50 | 0 | 5 mM | 100 | 71 |
| | | | | | MgSO4/ | | |
| | | | | | 10 mM | | |
| | | | | | Tris | | |
| 39 | 2.5 | 1000 | 100 | 10 | 5 mM | 100 | 96 |
| | | | | | MgSO4/ | | |
| | | | | | 10 mM | | |
| | | | | | Tris | | |
| 40 | 2.5 | 1000 | 100 | 30 | 5 mM | 100 | 100 |
| | | | | | MgSO4/ | | |
| | | | | | 10 mM | | |
| | | | | | Tris | | |
| 41 | 5 | 1000 | 50 | 5 | 5 mM | 100 | 79 |
| | | | | | MgSO4/ | | |
| | | | | | 10 mM | | |
| | | | | | Tris | | |
| 42 | 10 | 1000 | 50 | 20 | 5 mM | 100 | 63 |
| | | | | | MgSO4/ | | |
| | | | | | 10 mM | | |
| | | | | | Tris | | |
| 43 | 10 | 1000 | 100 | 30 | 5 mM | 100 | 106 |
| | | | | | MgSO4/ | | |
| | | | | | 10 mM | | |
| | | | | | Tris | | |
| 44 | 20 | 1000 | 50 | 10 | 5 mM | 100 | 51 |
| | | | | | MgSO4/ | | |
| | | | | | 10 mM | | |
| | | | | | Tris | | |
| 45 | 20 | 1000 | 50 | 30 | 5 mM | 100 | 84 |
| | | | | | MgSO4/ | | |
| | | | | | 10 mM | | |
| | | | | | Tris | | |
| 46 | 20 | 1000 | 100 | 10 | 5 mM | 100 | 65 |
| | | | | | MgSO4/ | | |
| | | | | | 10 mM | | |
| | | | | | Tris | | |
| 47 | 20 | 1000 | 100 | 30 | 5 ml | 100 | 139 |
| | | | | | MgSO4/ | | |
| | | | | | 10 mM | | |
| | | | | | Tris | | |
| 48 | 2.5 | 1250 | 50 | 0 | 5 mM | 2500 | 126 |
| | | | | | MgSO4/ | | |
| | | | | | 10 mM | | |
| | | | | | Tris | | |

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. The term “or” when used in the context of a listing of multiple options (e.g. “A, B, or C”) shall be interpreted to include any one or more of the options, unless the context clearly dictates otherwise.

Exemplary methods and materials are described herein, although methods and materials similar or equivalent to those described herein can also be used in the practice or testing of the present invention. The materials, methods, and examples are illustrative only and not intended to be limiting.

1. A biphasic mixture comprising a liquid phase and a solid phase, wherein:
a. the liquid phase is a solution comprising a molecule of interest and masked endotoxin; and
b. the solid phase is solid 1-dodecanol.

2. A method of unmasking endotoxin in a solution comprising a molecule of interest and masked endotoxin, the method comprising:
a. adding liquid phase 1-dodecanol to a solution containing the molecule of interest and the masked endotoxin; and
b. cooling the liquid phase 1-dodecanol and solution containing the molecule of interest to a temperature below 24° C., such that the 1-dodecanol solidifies and there is a remaining liquid portion and the solidified 1-dodecanol.

3. The method of claim 2, wherein the remaining liquid portion is “Liquid A”, and wherein the method further comprises adding a dispersant solution to Liquid A, to generate a mixture (“Liquid B”) comprising dispersant and Liquid A.

4. The method of claim 3, further comprising diluting a portion of Liquid B into a buffer comprising Ca²⁺ or Mg²⁺ ions to yield a sample ready for endotoxin testing, wherein the previously masked endotoxin is unmasked in the sample ready for endotoxin testing.

5. The method of claim 4, wherein the portion of Liquid B is diluted into the buffer comprising Ca²⁺ or Mg²⁺ ions at a ratio between a) 1 microliter Liquid B per 100 microliters Ca²⁺ or Mg²⁺ ions buffer and b) 1 microliter Liquid B per 3000 microliters Ca²⁺ or Mg²⁺ ions buffer.

6. A method of unmasking endotoxin in a solution comprising a molecule of interest and masked endotoxin, the method comprising:
a. adding liquid phase 1-dodecanol to a solution containing the molecule of interest and the masked endotoxin;
b. cooling the liquid phase 1-dodecanol and solution containing the molecule of interest to a temperature below 24° C., such that the 1-dodecanol solidifies and there is a remaining liquid portion (“Liquid A”) and the solid 1-dodecanol; and
c. diluting a portion of Liquid A into a buffer comprising Ca²⁺ or Mg²⁺ ions to yield a sample ready for endotoxin testing;
wherein the previously masked endotoxin is unmasked in the sample of step c.

7. The method of claim 4, wherein the buffer comprising Ca²⁺ or Mg²⁺ ions comprises Mg²⁺ ions.

8. The method of claim 7, wherein the buffer comprises MgSO₄.

9. The method of claim 8, wherein the buffer comprises 1, 5, or 10 mM MgSO₄.

10. The method of claim 4, wherein the sample ready for endotoxin testing further is tested for endotoxin via a Limulus Amebocyte Lysate (LAL) assay.

11. The method of claim 4, wherein a greater amount of endotoxin can be detected in the sample ready for endotoxin testing than from an otherwise identical corresponding solution containing the molecule of interest and the masked endotoxin that was not subject to the method of claim 4.

12. The method of claim 2, wherein the ratio of microliters of 1-dodecanol to microliters of solution comprising the molecule of interest and masked endotoxin is between a) 0.2 microliters 1-dodecanol per 10 microliters solution and b) 2 microliters 1-dodecanol per 10 microliters solution.

13. The method of claim 2, wherein the molecule of interest is a protein.

14. The method of claim 13, wherein the protein is an antibody.

15. The method of claim 14, wherein the antibody is tanezumab.

16. The method of claim 2 wherein the solution comprising a molecule of interest is a tanezumab drug product.

17. The method of claim 15, wherein the solution comprises 2.5 mg/ml, 5 mg/ml, 10 mg/ml, or 20 mg/ml tanezumab.

18. The method of claim 2, wherein the 1-dodecanol is ≥98% pure 1-dodecanol.

19. The method of claim 16, wherein the solution comprises 2.5 mg/ml, 5 mg/ml, 10 mg/ml, or 20 mg/ml tanezumab.
